(19) United States
(12) Patent Application Publication
(10) Pub. No.: US 2024/0430987 A1
(43) Pub. Date: Dec. 26, 2024

(54) USB POWERED HEATING PAD

(71) Applicant: Sunbeam Products, Inc., Atlanta, GA (US)

(72) Inventors: Lalit Mandal, Boca Raton, FL (US); Jill Walters, Delray Beach, FL (US)

(21) Appl. No.: 18/684,705

(22) PCT Filed: Sep. 9, 2022
(86) PCT No.: PCT/US2022/076200

§ 371 Date: Feb. 19, 2024

Related U.S. Application Data
(60) Provisional application No. 63/242,199, filed on Sep. 9, 2021.

(51) Int. Cl. H05B 3/00; H05B 1/02; H05B 3/34; H05B 3/56

(52) U.S. Cl. CPC H05B 3/0019; H05B 1/0272; H05B 3/342; H05B 3/56; H05B 2203/003; H05B 2203/016

(57) ABSTRACT

A USB heating pad is provided. Specifically. a USB heating pad of at least a conventional dimension that is capable of being heated up to a desired temperature within a desired timeframe. The USB heating pad includes a heat pad comprising a series of layers with a heating element sandwiched therein. The heating clement is further coupled to a USB connector and a controller. The heat pad is further connected to an engagement mechanism to be worn by a user.

### CROSS-REFERENCE TO RELATED APPLICATION

This application claims the benefit of the filing date of U.S. Provisional Application Ser. No. 63/242,199 filed Sep. 9, 2021, entitled, “USB Powered Heating Pad”, which is hereby incorporated by reference as if fully set forth herein.

### FIELD OF INVENTION

The invention refers to heating pads. More specifically, the invention includes heating pads that use a USB power source.

### BACKGROUND OF THE INVENTION

Clinical studies have shown that the application of heat may provide relief for muscle and joint pain. Chronic pain sufferers are open to and sometimes even prefer using non-oral pain relief methods, such as heat pads.

Current heating pads require a large amount of power to provide a high-level of heat because heat may be lost to the ambient air. Therefore, heating pads in use today are most often plugged into an outlet to provide a sufficiently high level of heat. These heating pads usually require that a user has at least 30 minutes and a plug, which means he or she may be limited to using the heating pad at home.

Despite the convenience offered by battery-powered heating pads compared to corded heating pads, battery powered heating pads have not been as successful in the market. Battery powered heating pads generally either require a large and unwieldy battery to provide sufficient power for a high-level of sustained heat, or the battery runs out of power too quickly.

Large batteries are heavy and uncomfortable to wear, reducing the likelihood that consumers will buy and use the heating pad. A heating pad that runs out of power too quickly or that does not get hot enough also reduces the likelihood that consumers will buy and use the heating pad. It is therefore desirable for a portable heating pad that delivers a high level of heat, while still being comfortable to wear.

With the rise of availability of personal computers, Universal Serial Bus (USB) has also been used as a power source for various applications. However, due to the challenging specifications associated with using USB, attempts to use USB as a power source for heating pads have failed.

Specifically, USB power standards generally limit a device wattage output. By way of example, conventional USB may provide up to 5 volts (V). In general, the specifications for a USB 1.0 and 2.0 standard downstream port deliver up to 500 milliamp (mA) or 0.5 A, while a USB 3.0 downstream port may provide up to 900 mA or 0.9 A, which translates into 4.5 watts. USB-C may deliver up to 3000 milliamp (mA) or 3.0 A, which translates into 15 watts.

Further, USB Battery Charging (BC) and Power Delivery (PD) include additional power-related specifications meant for higher power delivery aimed for faster charging of modern-day devices. As such, a USB BC 1.1 compatible downstream port may provide up to 1.5 A at 5V, which computes to 7.5 W, and a USB BC 1.2 compatible downstream port may provide up to 5 A at 5V, which computes to 25 W. On the other hand, a USB PD compatible downstream port may provide up to 20V. Nonetheless, most conventional devices, such as a generic USB port on a laptop, do not support the PD specification. Thus, there is a need for a full-size heating pad that may draw power from a USB source while maintaining desired performance characteristic of a traditional heating pad.

The power restraint of USB specification makes it challenging to meet desired performance characteristic of a heating pad. Namely, if a traditional heating pad is simply converted to draw power from a USB port, due to the power constraint, the heating pad either cannot reach desired heating temperature or would take an extended period of time to heat up to the desired temperature. Although it may be possible to meet the desired performance characteristic by reducing the size of the heating pad, a small heating pad is not as desirable because of its limited uses.

### SUMMARY OF THE INVENTION

A heating pad is provided that includes a flexible heat pad, a power button, a USB connector, and an engagement mechanism and which is heat-able using low voltage power from the USB connector. The heating pad is preferably of at least about 42 square-inches. The heat pad may have an anterior side and a posterior side, where the posterior side is adjacent to and faces the user and the anterior side of the heat pad faces away from the user when the heating pad is worn. The heat pad may include a series of flexible layers, allowing it to conform to the part of the user's body.

In one embodiment, the series of layers include a micromink layer that extends over the posterior side of the heat pad. Adjacent to the micromink layer, the heat pad may include a nylon layer. A wire may attach to the nylon layer.

The heat pad may also include a reflective layer that may reflect heat radiated towards the anterior side back towards the posterior side of the heat pad so that less heat is lost to the ambient air. The reflective layer may have an air gap on both of its sides in some embodiments.

The heat pad may further include a polyester batting fiber layer and a brushed polyester layer. The micromink layer and the brushed polyester layer are the visible outer layers of the heating pad on the posterior and anterior sides, respectively.

To use the heating pad, the user may place the heating pad onto the desired part of the body and wrap the engagement mechanism around the area to secure the heating pad in place. In one embodiment, the engagement mechanism is a strap that may engage the heating pad through a hook and loop engagement, though other mechanisms are foreseeable.

The wire pattern used to heat the heat pad may be specifically constructed to heat the heating pad to a desired temperature in a reasonable amount of time.

### BRIEF DESCRIPTION OF THE DRAWINGS

FIG. 1 illustrates a perspective view of a USB powered heating pad according to an example embodiment.

FIG. 2 illustrates a cross-sectional view of the USB powered heating pad of FIG. 1.

FIG. 3A illustrates another cross-sectional view of the USB powered heating pad of FIG. 1.

FIG. 3B illustrates a side view of a posterior side of the USB powered heating pad of FIG. 1.

FIG. 3C illustrates another cross-sectional view of the USB powered heating pad of FIG. 1.

FIG. 3D illustrates a top view of an engagement mechanism of the USB powered heating pad of FIG. 1.

FIG. 3E illustrates a side view of the engagement mechanism of FIG. 3D.

Before explaining the disclosed embodiment of the present invention in detail, it is to be understood that the invention is not limited in its application to the details of the particular arrangement shown, since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

While this invention is susceptible of embodiments in many different forms, there are shown in the drawings and will be described in detail herein specific embodiments with the understanding that the present disclosure is an exemplification of the principles of the invention. It is not intended to limit the invention to the specific illustrated embodiments. The features of the invention disclosed herein in the description, drawings, and claims may be significant, both individually and in any desired combinations, for the operation of the invention in its various embodiments. Features from one embodiment may be used in other embodiments of the invention.

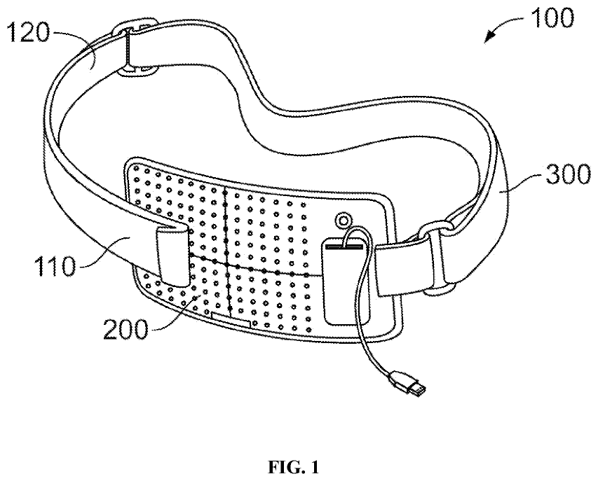

As shown in FIGS. 1-3E, the embodiments of this disclosure include a USB powered heating pad. Referring first to FIG. 1, a USB powered heating pad 100 may include a heat pad 200 and an engagement mechanism 300. The heating pad 100 may include an anterior side 110 and a posterior side 120. The anterior side 110 of the heating pad 100 may face outwardly from a wearer and is visible when the heating pad 100 is worn. The posterior side 120 of the heating pad 110 may be the side adjacent to the wearer when the heating pad 110 is worn.

In one embodiment, the engagement mechanism 300 may be a strap adjacent to the heat pad 200. The engagement mechanism 300 may include at least one hook, while the anterior side 110 of heat pad 200 may be made of material that includes at least one loop to selectively engage the at least one hook on the engagement mechanism 300 in a hook and loop engagement, as would be understood. For example, the material may be brushed polyester or 3D spacer mesh fabric. The material on the anterior side 110 of the heat pad 200 allows the engagement mechanism 300 to selectively engage the heat pad 200 through a hook and loop engagement. In alterative embodiments, the engagement mechanism 300 may be other types of engagement mechanism other than a hook and loop-type mechanism.

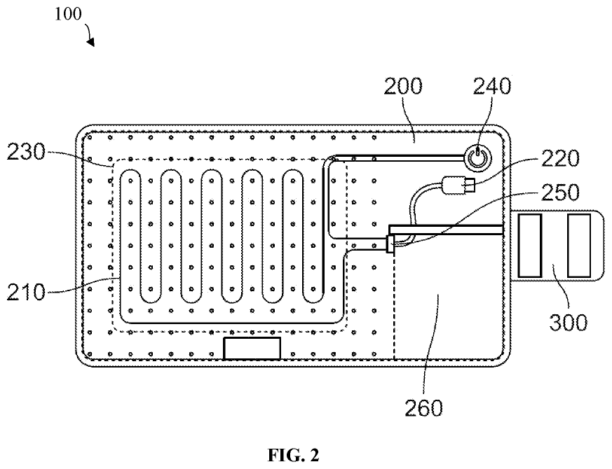

FIG. 2 illustrates a cross-sectional diagram of the heat pad 200. The heat pad 200 may be at least about 15 square-inches, but more typically may be at least about 20 square-inches or at least about 30 square-inches, or in some embodiments may be at least 40 square-inches or more. In some embodiments, the heat pad 200 may be rectangular and at least 6 by 8 inches, or in other embodiments may be at least 7 by 8 inches. The heat pad 200 may include a heating element 210 running therethrough. The heating element 210 may be electrically coupled to a USB connector 220 on a first end. The USB connector 220 may be any type of conventional USB connector (such as USB type A, B, C, mini USB, or micro USB). In an embodiment, the USB connector 220 may be a USB type C female connector. In some embodiments, the heat pad 200 may be designed to heat up properly with a power draw of about 5V and about 2 A, i.e., about 10 watts, or less than about 20 watts. Other power draws based on various combinations of amps with 5V are also contemplated.

In some embodiments, the heating element 210 may be a low voltage resistive wire having a first length and a first wire diameter, resulting in a first total resistance. The wire may be less than 2 millimeter (mm) in diameter, and more typically less than 1.5 mm, but generally between 0.5 to 1.5 mm, or 0.8 to 1.2 mm in diameter. The length of the wire, as well as the wire diameter, may impact the heating characteristic of the heat pad 200. In some embodiments, the length of the wire may be about five times the area of the heated pad in cm. For example, a heated area of 20 by 10 centimeters may have an area of 200 square-centimeters and a wire length of about 1 meter (m). If the wire were too long, given the same wire diameter, it would take too long to heat up the heat pad 200 with a given power constraint. Alternatively, if the wire were too short, then the heated area of the heat pad 200 would be too small. Thus, properly balancing the geometry and dimension of the heating element 210 together with the desired dimension of the heat pad 200 may increase the functionality and desirability of the heating pad 100. Typically, the first total resistance may be about 1 to 2 Ohms, more typically 1.5 to 1.8 or about 1.8 Ohms when measured as a resistance of the total heat pad when measured at 25 degree Celsius or less.

In an example embodiment, the desired heated area 230 may be seven (7) inches by eight (8) inches, thus having an effective heated area of 56 square-inches (or about 360 square-centimeters). Moreover, it may be desired that the heat pad 200 may heat up to a first target temperature within a first target time frame. In this example embodiment, the first target temperature may be about 145 F, and the first target time frame may be less than sixteen (16) minutes from room temperature. Moreover, the power draw from the USB connector 220 may be limited to 5V and 2 A. Using these requirements as an example, the heating element 210 may be a low voltage resistive wire that is 1.8 meters (m) long, having a wire diameter that ranges from 0.8 millimeter (mm) to 1.2 mm and wherein the pad has a total resistance of about 1.8 ohms. As illustrated in FIG. 2, the heating element 210 may be laid out in a generally square-wave or sinusoidal pattern.

The heating element 210 may further be electrically coupled to a controller 240. In an embodiment, the controller 240 may be a push button controller located on the heat pad 200. In some embodiments, the controller 240 may include one or more indicators (such as LED or other displays). In certain embodiments, the controller 240 may control the heating element 210 so that the heating pad 100 may have more than one heat settings.

Using the above embodiment as an example, the controller 240 may be configured to produce three different heat settings: low, medium, and high. Given that the maximum heat output at the example embodiment may be about 145 (−15/+25)F, such maximum heat output may be set as high heat setting. By controlling a duty cycle, i.e., on and off of the heating element 210, the controller 240 may also control the heating element 210 to produce a medium setting at about 130 (+/−10)F, and a low setting at about 110 (+/−15)F. It should be noted that the aforementioned settings are illustrative only, and by regulating duty cycle, the controller 240 may be configured to produce a variety of different heat settings.

The heat pad 200 may also include an aperture or opening 250 so that the USB connector 220 may protrude out of the various layers (described below) of the heat pad 200. In an example embodiment, the USB connector 220 may protrude out of the heat pad 200 from the anterior side 110. Moreover, a pocket or pouch 260 may be provided on a surface of the heat pad 200 that may function as a small storage for the cord of the USB connector 220.

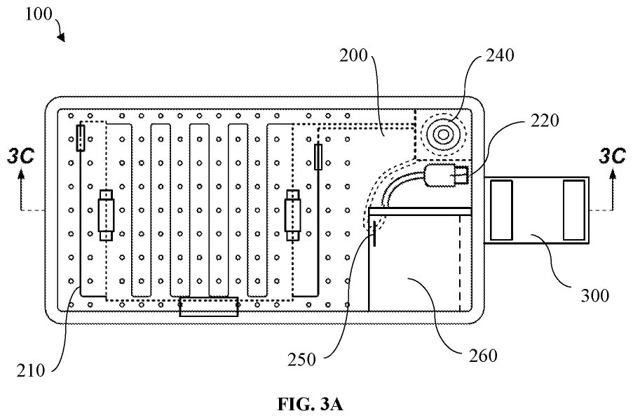

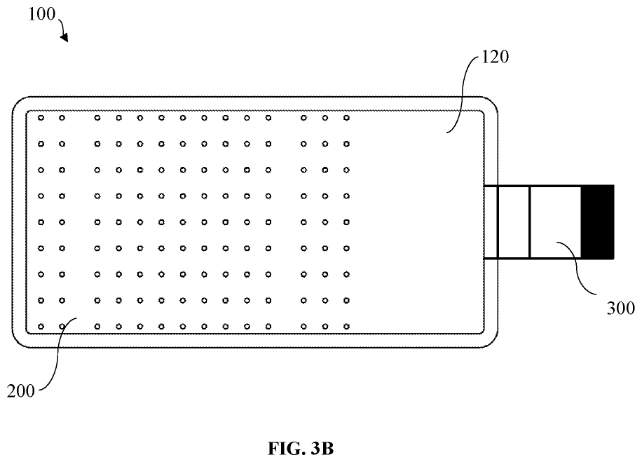

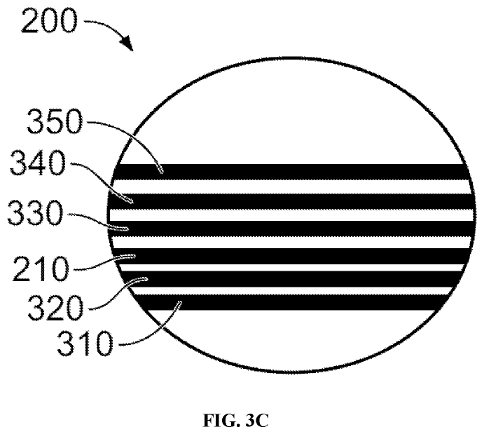

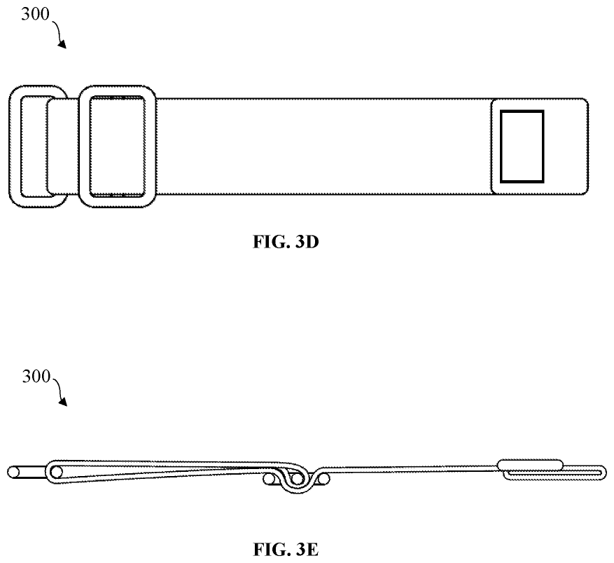

Referring now to FIG. 3A-3E, various details of the heating pad 100 are illustrated. In some embodiments, the heat pad 200 may comprise a plurality of flexible layers. In an example embodiment, as shown in FIG. 3C, the heat pad 200 may comprise a series of five layers in addition to the heating element 210 sandwiched therein.

The series of layers may include a first layer 310 that covers the posterior side 120 of the heat pad 200 and is positioned adjacent to the user when the heating pad 100 is worn. Because the first layer 310 is the layer that comes into contact with the user's skin or clothing, it preferably provides a comfortable surface that does not irritate the skin of the user when the heating pad 100 is worn. The layer 310 should also able to transfer heat. In some embodiments, the first layer 310 may be a micromink layer. The first layer 310 can, for example, have a weight near 200 g/m2.

The heat pad 200 may further include a second layer 320 that provides a surface for the heating element 210 to be attached to. The second layer 320 can, for example, be nylon scrim layer. The nylon fabric may have a weight of around 50 g/m2.

The heat pad 200 may also include a third layer 330 with the second layer 320 preferably located and positioned in between the first layer 310 and the third layer 330. The third layer 330 may be any appropriate type of fabric or material that may reflect heat towards the posterior side 120 of the heat pad 200 so that the posterior side 120 of the heat pad 200 is warmer than the anterior side 110 of the heat pad 200. In one embodiment, the third layer 330 may comprise hollow, polyester fibers needle punched through both a nonwoven substrate and a reflective metalized polyester film. The third layer 330 may further be surrounded on both sides by an air gap or air space. The reflective third layer 330 may have a weight of approximately 150 g/m2.

The heat pad 200 may further include a fourth layer 340 located and positioned on the anterior side 120 of the third layer 330. The third layer 330 may therefore preferably located and positioned in between the second layer 320 and the fourth layer 340. The fourth layer 340 may be a polyester batting fiber layer having a weight of about 150 g/m2.

Finally, the heat pad 200 may include a fifth layer 350, which may be a brushed polyester layer in one embodiment. The brushed polyester layer may have a weight of 130 g/m2. The visible outer layers of the heating pad 100 include the first layer 310 and the fifth layer 350, where only the fifth layer 350 is visible when the heating pad 100 is worn.

In practice, the user may place the heating pad 100 onto the desired part of the body, wrap the engagement mechanism 300 around the area, and secure the heating pad 100 through the engagement mechanism 300. The user may the plug the USB connector 220 into an appropriate USB port, such as ones found on a laptop or personal computers, a PowerBank, or be plugged into an electrical outlet through an adaptor, with or without a USB extension cord. The user may then turn on the heating pad 100 and adjust the temperature through the controller 240.

From the foregoing, it will be seen that the various embodiments of the present invention are well adapted to attain all the objectives and advantages hereinabove set forth together with still other advantages which are obvious and which are inherent to the present structures. It will be understood that certain features and sub-combinations of the present embodiments are of utility and may be employed without reference to other features and sub-combinations. Since many possible embodiments of the present invention may be made without departing from the spirit and scope of the present invention, it is also to be understood that all disclosures herein set forth or illustrated in the accompanying drawings are to be interpreted as illustrative only and not limiting. The various constructions described above and illustrated in the drawings are presented by way of example only and are not intended to limit the concepts, principles and scope of the present invention.

Many changes, modifications, variations and other uses and applications of the present invention will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A heating pad comprising:
a heat pad coupled to an engagement mechanism, the heat pad including a heating element coupled to a USB connector and a controller;
wherein the controller is configured to generate a first duty cycle for the heating element so that the heat pad outputs a first temperature after being on for a first time frame; and
wherein the USB connector is powered at 5V.

2. The heating pad of claim 1, wherein the first temperature is about 145 F, and the first time frame is less than 16 minutes.

3. The heating pad of claim 1, wherein the head pad includes a heating area of about 56 square inches.

4. The heating pad of claim 1, wherein the heating area is about 7 inches wide and 8 inches in length.

5. The heating pad of claim 1, wherein the heating element is a low voltage resistive wire.

6. The heating pad of claim 5, wherein the low voltage resistive wire is at least 1.8 meters in length and has a diameter of about 1 millimeter.

7. The heating pad of claim 1, wherein the controller is further configured to generate a second duty cycle for the heating clement so that the heat pad may output a second temperature.

8. The heating pad of claim 7, wherein the controller is further configured to generate a third duty cycle for the heating clement so that the heat pad may output a third temperature.
